(19) United States
(12) Patent Application Publication
(10) Pub. No.: US 2026/0069673 A1
(43) Pub. Date: Mar. 12, 2026

(54) BIVALENT INACTIVATED EV71-CA16 VACCINE, METHOD FOR PREPARING THE SAME, AND USE THEREOF

(71) Applicant: AIM ACTION BIOPHARM CO., LTD, TAIZHOU, JIANGSU (CN)

(72) Inventors: ERXIA YANG, TAIZHOU (CN); LI JIANG, TAIZHOU (CN); BIYUN LIU, TAIZHOU (CN); HAORAN ZHANG, TAIZHOU (CN); ZHIWEI ZHANG, TAIZHOU (CN); LIANGYU CHEN, TAIZHOU (CN); YUAN JI, TAIZHOU (CN)

(73) Assignee: AIM ACTION BIOPHARM CO., LTD, TAIZHOU, JIANGSU (CN)

(21) Appl. No.: 19/106,862

(22) PCT Filed: Oct. 27, 2023
(86) PCT No.: PCT/CN2023/126963

§ 371 Date: Feb. 26, 2025

(30) Foreign Application Priority Data
Dec. 13, 2022 (CN) 202211592176.1

(51) Int. Cl. A61K 39/125; A61K 39/00; A61K 39/295; A61K 39/39; A61P 31/14; C12N 7/02; C12N 7/04

(52) U.S. Cl. CPC A61K 39/125; A61K 39/295; A61K 39/39; A61P 31/14; C12N 7/02; C12N 7/045; A61K 2039/5252; A61K 2039/55505; A61K 2039/70; C12N 2770/32334; C12N 2770/32351; C12N 2770/32361; C12N 2770/32371

(57) ABSTRACT

The present invention relates to the technical field of vaccine preparation, and particularly, to a bivalent inactivated EV71-CA16 vaccine, a method for preparing the same, and use thereof. The present invention implements the preparation by means of separately adsorbing a monovalent viral stock solution containing an EV71 antigen and a monovalent viral stock solution containing a CA16 antigen with an adjuvant and then mixing same, thereby improving the content of the EV71 antigen and the CA16 antigen in the vaccine substance and greatly improving the adsorption rate and recovery rate of EV71 and CA16 antigens. Moreover, the bivalent EV71-CA16 viral antigen can cause positive seroconversion and up-regulation of the corresponding neutralizing antibody and induce a higher serum antibody level, thus facilitating the preparation of the bivalent inactivated EV71-CA16 vaccine and preventing hand-foot-and-mouth disease.

The present application claims the right of priority for Chinese patent application No. CN202211592176.1, filed with the China National Intellectual Property Administration on Dec. 13, 2022 and entitled “BIVALENT INACTIVATED EV71-CA16 VACCINE, METHOD FOR PREPARING THE SAME, AND USE THEREOF”, which is incorporated herein by reference in its entirety.

### TECHNICAL FIELD

The present disclosure relates to the technical field of vaccine preparation, and particularly, to a bivalent inactivated EV71-CA16 vaccine, a method for preparing the same, and use thereof.

### BACKGROUND

Hand-foot-and-mouth disease is an acute infectious disease caused by enteroviruses, which commonly occurs in infants and young children, and the annual incidence has been rising in the past two decades, and has formed a serious epidemiological trend. Hand-foot-and-mouth disease can cause herpes in the parts such as hands, feet and mouth. A small number of sick children may suffer from complications such as myocarditis, pulmonary edema and aseptic meningoencephalitis, and few severe ones may even die if the disease progresses rapidly.

There are more than 20 enteroviruses that cause hand-foot-and-mouth disease, with Coxsackie virus type A16 (CA16) and Enterovirus type 71 (EV71) being the most common. At present, EV71 vaccine has been marketed, but the research progress of CA16 vaccine is still slowly. The existing monovalent CA16 vaccine has low content, adsorption rate and recovery rate of antigen, which cannot exert its immunization effectiveness. Although the symptoms of hand-foot-and-mouth disease caused by CA16 are mild, the number of cases caused by CA16 is gradually increasing in recent years, and a small number of severe cases can also be caused. Although EV71 vaccine has effectively reduced the incidence of severe hand-foot-and-mouth disease and death, it lacks cross-protection and therefore cannot resist hand-foot-and-mouth disease caused by other pathogens including CA16. To completely control the occurrence and development of hand-foot-and-mouth disease in children, CA16 should also be the subject of significant attention.

Currently, there are also a large number of studies on bivalent EV71 and CA16 vaccines in the prior art, for example, the prior art “Study on Immunogenicity in Mice Induced by Bivalent Inactivated EV71 and CA16 vaccine” (X WANG, F GAO, QY MAO et al., Study on Immunogenicity in Mice Induced by Bivalent Inactivated EV71 and CA16 vaccine [J]. Progress in Microbiology and Immunology, 2014, 42 (2): 1-1) discloses an bivalent inactivated EV71 and CA16 vaccine and the study on the immunogenicity induced by the vaccine, where although the resulting bivalent inactivated EV71 and CA16 vaccine can induce a neutralizing antibody response level similar to that of monovalent vaccines, the prepared vaccine has a relatively low immunogenicity and poor stability.

### SUMMARY

An objective of the present disclosure is to provide a bivalent inactivated EV71-CA16 vaccine, a method for preparing the same, and the use thereof, where the bivalent inactivated EV71-CA16 vaccine has a more stable and effective immune response than monovalent EV71 vaccines or monovalent CA16 vaccines, and can induce a higher serum antibody level.

The present disclosure provides a method for preparing a bivalent inactivated EV71-CA16 vaccine, which method includes the steps of:
separately mixing and adsorbing a monovalent EV71 stock solution and a monovalent CA16 stock solution with an adjuvant to obtain a monovalent EV71-adjuvant adsorption product and a monovalent CA16-adjuvant adsorption product, respectively;
mixing and stirring the monovalent EV71-adjuvant adsorption product and the monovalent CA16-adjuvant adsorption product, and mixing the resulting mixture with a protectant to obtain the bivalent inactivated EV71-CA16 vaccine.

In some embodiments, the adjuvant includes aluminum hydroxide, and the mass concentration of the adjuvant in the bivalent inactivated EV71-CA16 vaccine is 0.25 to 1.0 mg/mL.

In some embodiments, the protectant includes glycine, and the mass concentration of the glycine in the bivalent inactivated EV71-CA16 vaccine is 3 to 10 mg/mL.

In some embodiments, the time of the stirring is 30 min.

In some embodiments, prior to mixing with the protectant, further included is: diluting the resulting mixture using a buffer.

In some embodiments, the buffer includes a PBS buffer, and the PBS buffer has a concentration of 2 mM and a pH value of 7.00 to 7.40.

The present disclosure further provides a bivalent inactivated EV71-CA16 vaccine, which is prepared using the preparation method of the above technical solution.

In some embodiments, the content of the EV71 antigen and the content of the CA16 antigen in the bivalent inactivated EV71-CA16 vaccine are both 500 to 2000 U/mL.

In some embodiments, the dose of the EV71 antigen and the dose of the CA16 antigen in the bivalent inactivated EV71-CA16 vaccine are both 100 to 400 U/dose.

The present disclosure further provides the use of the bivalent inactivated EV71-CA16 vaccine prepared by the preparation method of the above technical solution for the preparation of a kit for preventing hand-foot-and-mouth disease.

### Beneficial Effects

The present disclosure provides a method for preparing a bivalent inactivated EV71-CA16 vaccine. The present disclosure implements the preparation by means of separately adsorbing a monovalent viral stock solution containing an EV71 antigen and a monovalent viral stock solution containing a CA16 antigen with an adjuvant and then mixing same, thereby improving the content of the EV71 antigen and the CA16 antigen in the vaccine substance and greatly improving the adsorption rate and recovery rate of EV71 and CA16 antigens. Moreover, the bivalent EV71-CA16 viral antigen can cause positive seroconversion and up-regulation of the corresponding neutralizing antibody and induce a higher serum antibody level, thus having a more stable and effective immune response. In addition, the bivalent inactivated EV71-CA16 vaccine prepared using the above preparation method has the advantages of low inoculation amount and few adverse reactions, and can induce a high-level antibody response after immunization.

### BRIEF DESCRIPTION OF THE DRAWINGS

To describe the technical solutions in the examples of the present disclosure or in the prior art more clearly, the accompanying drawings required for the examples will be briefly described below:

FIG. 1 shows the recovery rates of antigens in bivalent inactivated EV71-CA16 vaccines of Example 1 and Comparative examples 1 to 3;

FIG. 2 shows the OD values of free antigens in the supernatants of bivalent inactivated EV71-CA16 vaccines of Example 1 and Comparative Examples 1-3:

FIG. 3 shows the statistical results of potencies of neutralizing antibodies (GMT value) against CA16 in the sera of mice immunized with bivalent inactivated EV71-CA16 vaccines of Example 1 and Comparative examples 1 to 3:

FIG. 4 shows the statistical results of potencies of neutralizing antibodies (GMT value) against EV71 in the sera of mice immunized with bivalent inactivated EV71-CA16 vaccines of Example 1 and Comparative examples 1 to 3:

FIG. 5 shows the statistical results of potencies of neutralizing antibodies (GMT value) against EV71 in the sera of mice immunized with bivalent inactivated EV71-CA16 vaccine of Example 1 and monovalent inactivated EV71 vaccine of Comparative example 4:

FIG. 6 shows the statistical results of potencies of neutralizing antibodies (GMT value) against CA16 in the sera of mice immunized with bivalent inactivated EV71-CA16 vaccine of Example 1 and monovalent inactivated CA16 vaccine of Comparative example 5.

### DETAILED DESCRIPTION OF THE EMBODIMENTS

The present disclosure provides a method for preparing a bivalent inactivated EV71-CA16 vaccine, which method includes the steps of:
separately mixing and adsorbing a monovalent EV71 stock solution and a monovalent CA16 stock solution with an equal amount of adjuvant to obtain a monovalent EV71-adjuvant adsorption product and a monovalent CA16-adjuvant adsorption product, respectively:
mixing and stirring the monovalent EV71-adjuvant adsorption product and the monovalent CA16-adjuvant adsorption product, and mixing the resulting mixture with a protectant to obtain the bivalent inactivated EV71-CA16 vaccine.

In the present disclosure, a monovalent EV71 stock solution and a monovalent CA16 stock solution are separately mixed and adsorbed with an adjuvant to obtain monovalent EV71-adjuvant adsorption product and a monovalent CA16-adjuvant adsorption product, respectively. In the present disclosure, there is no specific limitation on the process of mixing and adsorbing, as long as mixing and adsorbing well known to a person skilled in the art are used. In some embodiments of present application, both the monovalent EV71 stock solution and the monovalent CA16 stock solution are inactivated viral stock solutions. The monovalent EV71 stock solution of the present disclosure contains Enterovirus type 71. In some embodiments, the virus strain of the Enterovirus type 71 includes FY23 or TZ06-N-M, preferably TZ06-N-M. The TZ06-N-M is deposited in the China General Microbiological Culture Collection Center on Oct. 16, 2018 (abbreviated as CGMCC: address: Institute of Microbiology. Chinese Academy of Sciences. No. 3, Yard 1, Beichen West Road. Chaoyang District. Beijing: postcode: 100101) with the deposit number of CGMCC No. 16684, and published in Chinese patent 201811294631.3 (date of publication: Apr. 12, 2019). The monovalent CA16 stock solution of the present disclosure contains Coxsackie virus type A16: in some embodiments, the virus strain of the Coxsackie virus type A16 includes KM/M08 or G20, preferably G20. In some embodiments, the method for preparing the monovalent EV71 stock solution of the present disclosure includes the steps of:
inoculating a virus strain of Enterovirus type 71 onto monolayer cultured cells for culture to obtain a virus solution:
performing ultrafiltration, concentration and sterilization on the virus solution to obtain a concentrated virus solution:
performing purification, inactivation and concentration on the concentrated virus solution to obtain the monovalent EV71 stock solution.

In some embodiments, the present disclosure is preferably inoculating the virus strain of Enterovirus type 71 onto monolayer cultured cells for culture to obtain a virus solution. In some embodiments, the inoculation amount of the virus strain of the present disclosure is 0.1 to 0.25 MOI, preferably 0.1 MOI. In some embodiments, the cultured cells of the present disclosure include KMB17 cells. In some embodiments, the temperature of the culture of the present disclosure is 36.5° C. to 37.5° C., preferably 37° C.; and the time of the culture is 36 to 80 h, preferably 69 h. When the culture is completed, the cultured cells is in a pathological state of cell shrinking.

In the present disclosure, a resulting virus solution is conducted to ultrafiltration. In some embodiments, the present disclosure is sequentially using a depth filter and a sterilizing filter to perform the ultrafiltration. In some embodiments of the present disclosure, the depth filter has a filtration accuracy of 0.45 μm; and the sterilizing filter has a filtration accuracy of 0.45 μm.

In some embodiments, a concentration is performed on a resulting filtrate from the ultrafiltration. In some embodiments, the concentration of the present disclosure includes performing concentration using an ultrafiltration membrane pack with a molecular weight cut-off of 100 KD, and the filtrate is concentrated 60 times. In some embodiments, the present disclosure further includes rinsing the filter member of the ultrafiltration membrane pack using a PBS buffer, and the concentration of the PBS buffer is 2 mmol/L.

In some embodiments of the present disclosure, after the concentration, it further includes subjecting the resulting concentrated liquid after the concentration to a 0.22 μm sterilizing filter to obtain a concentrated virus solution.

In some embodiments of the present disclosure, after the concentrated virus solution is obtained, purification is performed on the concentrated virus solution. In some embodiments of the present disclosure, the purification is performed using a Sepharose 6FF pre-packed column; and the column height of the pre-packed column is 85 cm. In some embodiments of the present disclosure, the loading amount of the concentrated virus solution is 5% CV, and the flow rate is 5 mL/min. In some embodiments of the present disclosure, sampling is performed between peak 1 and peak 2, and the time of the sampling is 60 min.

In some embodiments of the present disclosure, after the purification, inactivation is performed on a resulting purified virus solution from the purification. In some embodiments of the present disclosure, the inactivation is conducted by mixing formaldehyde with the purified virus solution and performing a reaction for 12 days. In some embodiments of the present disclosure, the final concentration of the formaldehyde in the purified virus solution is 90 μg/mL.

In some embodiments of the present disclosure, after the inactivation, the concentration is performed on the inactivated virus solution to obtain the monovalent EV71 stock solution. In some embodiments of the present disclosure, the conditions of the concentration are the same as the conditions of the concentration described above, which will not be repeated here.

In some embodiments of the present disclosure, the method for preparing the monovalent CA16 stock solution is the same as the method for preparing the monovalent EV71 stock solution, which will not be repeated here.

In some embodiments of the present disclosure, the initial content of the antigen in the monovalent EV71 stock solution and the initial content of the antigen in the monovalent CA16 stock solution are ≥4000 U/mL.

In some embodiments of the present disclosure, the adjuvant includes aluminum hydroxide. In the present disclosure, there is no specific limitation on the source of the aluminum hydroxide, as long as aluminum hydroxide from a conventional source in the art is used. In some embodiments of the present disclosure, the mass concentration of the aluminum hydroxide in the bivalent inactivated EV71-CA16 vaccine is 0.25 to 1.0 mg/mL, preferably 0.5 to 0.75 mg/mL, more preferably 0.5 mg/mL. In some embodiments of the present disclosure, an equal amount of adjuvant is mixed with a monovalent EV71 stock solution and a monovalent CA16 stock solution, respectively. In the present disclosure, there is no specific limitation on the actual amount of adjuvant, as long as the mass concentration of the adjuvant in the bivalent inactivated EV71-CA16 vaccine is ensured.

In some embodiments of the present disclosure, the protectant includes glycine. In some embodiments of the present disclosure, the mass concentration of the protectant in the bivalent inactivated EV71-CA16 vaccine is 3 to 5 mg/mL, preferably 3 mg/mL.

In the present disclosure, after the monovalent EV71-adjuvant adsorption product and the monovalent CA16-adjuvant adsorption product are obtained, the monovalent EV71-adjuvant adsorption product and the monovalent CA16-adjuvant adsorption product are mixed and stirred. In some embodiments of the present disclosure, the time of the stirring is 30 min; and the temperature of the stirring is room temperature. In some embodiments of the present disclosure, the stirring is performed using a magnetic stirrer to uniformly mix the monovalent EV71-adjuvant adsorption product and the monovalent CA16-adjuvant adsorption product.

In the present disclosure, after the stirring, the resulting mixture is mixed with a protectant to obtain the bivalent inactivated EV71-CA16 vaccine. In some embodiments of the present disclosure, it further includes on the basis of the desired potencies of an EV71 antigen and a CA16 antigen in a bivalent inactivated EV71-CA16 vaccine, diluting the resulting mixture using a buffer and then mixing the diluted mixture with a protectant to obtain bivalent inactivated EV71-CA16 vaccines having different potencies. The buffer of the present disclosure is a PBS buffer, and the PBS buffer has a concentration of 2 mM; and the PBS buffer has a pH value of 7.00 to 7.40. In the present disclosure, there is no specific limitation on the amount of the buffer, and the amount of the buffer is jointly determined by the initial concentrations of the viral stock solutions in the bivalent inactivated EV71-CA16 vaccine preparation system and the potencies of the EV71 antigen and the CA16 antigen in the bivalent inactivated EV71-CA16 vaccine. For example, in the example of the present disclosure, the initial concentrations of the monovalent EV71 stock solution and the monovalent CA16 stock solution are 5928 U/mL and 4658 U/mL, respectively: in the bivalent inactivated EV71-CA16 vaccine, the potencies of the EV71 antigen and the CA16 antigen are both 1000 U/mL, the mass concentration of the aluminum hydroxide is 0.5 mg/mL, and the mass concentration of the glycine is 3 mg/mL; and the amount of 2 mM PBS buffer is 11.1 mL.

The present disclosure further provides a bivalent inactivated EV71-CA16 vaccine prepared using the preparation method of the above technical solution. In some embodiments of the present disclosure, the potencies of the EV71 antigen and the CA16 antigen in the bivalent inactivated EV71-CA16 vaccine are both 100 to 400 U/dose, preferably 200 U/dose. Compared with the preparation method of mixing a monovalent EV71 stock solution and a monovalent CA16 stock solution and then adsorbing the resulting mixture with an adjuvant, the preparation method of separately adsorbing a monovalent viral stock solution containing an EV71 antigen and a monovalent viral stock solution containing a CA16 antigen with an adjuvant and then mixing the resulting solutions of present disclosure can improve the content of the EV71 antigen and the CA16 antigen in the vaccine substance and greatly improve the adsorption rate and recovery rate of EV71 and CA16 antigens. Moreover, the bivalent EV71-CA16 viral antigen can cause positive seroconversion and up-regulation of the corresponding neutralizing antibody and induce a higher serum antibody level.

On the basis of the above advantages of the bivalent inactivated EV71-CA16 vaccine of the present disclosure, the present disclosure further provides the use of the bivalent inactivated EV71-CA16 vaccine of the above technical solution for the preparation of a kit for preventing hand-foot-and-mouth disease. The bivalent inactivated EV71-CA16 vaccine provided by the present disclosure has a protective effect against hand-foot-and-mouth disease caused by viruses including EV71 and CA16, a broader range of prevention, a stronger immunogenicity, a high stability, low amount, few adverse reaction and a better effect, and can overcome the difficulty that the existing inactivated vaccine against hand-foot-and-mouth disease cannot exert immunization effectiveness.

To further illustrate the present disclosure, the technical solutions provided by the present disclosure will be described in detail in conjunction with the accompanying drawings and examples below, which cannot be construed as limiting the scope of protection of the present disclosure.

### Example 1 and Comparative Examples 1 to 3

A method for preparing a bivalent inactivated EV71-CA16 vaccine, involving the steps of:

TABLE 1
Method for dilute preparation of bivalent inactivated EV71-CA16 vaccine and adjuvant

| No. | Order of adding aluminium hydroxide adjuvant |
|---|---|
| Example 1 | Separately adsorbing an EV71 stock solution and a CA16 stock solution |
| | with an Al(OH)₃ adjuvant and then mixing the resulting solutions |
| Comparative | Mixing a monovalent EV71 stock solution and a monovalent CA16 stock solution |
| example 1 | and then adsorbing the resulting mixture with an Al(OH)₃ adjuvant |
| Comparative | Adsorbing a monovalent EV71 stock solution with an Al(OH)₃ adjuvant |
| example 2 | and then adding a monovalent CA16 stock solution |
| Comparative | Adsorbing a monovalent CA16 stock solution with an Al(OH)₃ adjuvant |
| example 3 | and then adding a monovalent EV71 stock solution |

TABLE 2
Formula of bivalent inactivated EV71-CA16
vaccine (0.5 mg/mL Al(OH)₃ adjuvant)

| | Initial | | | Final | |
|---|---|---|---|---|---|
| | concentration | | Amount | concentration | |
| Component | (mL⁻¹) | | (mL) | (mL⁻¹) | |
| Monovalent EV71 | 5928 | U | 3.37 | 1000 | U |
| stock solution | | | | | |
| Monovalent CA16 | 4658 | U | 4.29 | 1000 | U |
| stock solution | | | | | |
| Al(OH)₃ | 13.5 | mg | 0.74 | 0.5 | mg |
| PBS buffer | 2 | mM | 11.1 | / | |
| Glycine | 120 | mg | 0.5 | 3 | mg |

| | | |
|---|---|---|
| Total volume | 20 | / |

### 1) Step of Preparing the Bivalent Inactivated EV71-CA16 Vaccine of Example 1:

The bivalent inactivated EV71-CA16 vaccine was prepared according to the dilute preparation method of “Example 1” in Table 1: the monovalent EV71 stock solution (batch number: TZ201702) and the monovalent CA16 stock solution (batch number: GX201702) were separately and accurately pipetted into different 15 mL centrifuge tubes according to the formula in “Table 2”, an Al(OH)₃ adjuvant was added in an amount of ½ of the amount in “Table 2”, and the resulting mixture was fully and uniformly mixed, and magnetically stirred at room temperature for 30 min to uniformly mix the two monovalent virus-aluminum adsorption products: 2 mM PBS buffer was supplemented, a glycine mother liquor was further supplemented according to the amount, and the resulting mixture was fully and uniformly mixed; the uniformly mixed sample was sub-packaged to obtain the bivalent inactivated EV71-CA16 vaccine, where the potencies of the EV71 antigen and the CA16 antigen in the bivalent inactivated EV71-CA16 vaccine were both 200 U/dose; and the vaccine was placed in a refrigerator at 2° C. to 8° C. for later use.

The step of preparing the monovalent EV71 stock solution was as follows:

With regard to the preparation process, a TZ06-N-M virus species was inoculated onto KMB17 cells grown as a monolayer at 0.1 MOI, and cultured at 37° C.±0.5° C. for 69 h to obtain the virus solution, at which time the KMB17 cells were in a pathological state of complete cell shrinking: the virus solution was filtered sequentially using a 0.45 μm depth filter and a 0.45 μm sterilizing-grade filter, then concentrated using an ultrafiltration membrane pack with a molecular weight cut-off of 100 KD for 60 times, the filter member of the ultrafiltration membrane pack was rinsed with 2 mmol/L PBS buffer, and filtration was performed using a 0.22 μm sterilizing-grade filter to obtain a concentrated virus solution: purification was performed using Sepharose 6FF packing with a column height of 85 cm, the concentrated virus solution was loaded at 5% CV at a loading flow rate of 5 mL/min, and sampling was performed when peak 1 dropped to the peak base to obtain a purified virus solution, with a total collection time of 60 min; the purified virus solution was inactivated for 12 days by adding formaldehyde with a final concentration of 90 μg/mL to obtain an inactivated purified virus solution; and the inactivated purified virus solution was concentrated using an ultrafiltration membrane pack with a molecular weight cut-off of 100 KD, and filtered using a 0.22 μm sterilizing-grade filter to obtain the monovalent EV71 stock solution.

The step of preparing the monovalent CA16 stock solution was the same as the step of preparing the monovalent EV71 stock solution, except that the TZ06-N-M virus species was replaced with G20.

The method for preparing the monovalent EV71 stock solution and the method for preparing the monovalent CA16 stock solution in the following examples and comparative examples were performed as in Example 1, which will not be repeated here.

### 2) Step of Preparing the Bivalent Inactivated EV71-CA16 Vaccine of Comparative Example 1:

The bivalent inactivated EV71-CA16 vaccine was prepared according to the dilute preparation method of “Comparative example 1” in Table 1: the monovalent EV71 stock solution (batch number: TZ201702) and the monovalent CA16 stock solution (batch number: GX201702) were separately and accurately pipetted into the same 50 mL centrifuge tube according to the formula in “Table 2”, and fully and uniformly mixed, and added with an Al(OH)₃ adjuvant according to the amount, and the resulting mixture was magnetically stirred at room temperature for 30 min: supplemented with 2 mM PBS buffer, further supplemented with a glycine mother liquor according to the amount, and the resulting mixture was fully and uniformly mixed: a resulting uniformly mixed sample was sub-packaged to obtain the bivalent inactivated EV71-CA16 vaccine, where the potencies of the EV71 antigen and the CA16 antigen in the bivalent inactivated EV71-CA16 vaccine were both 200 U/dose; and the vaccine was placed in a refrigerator at 2° C. to 8° C. for later use.

### 3) Step of Preparing the Bivalent Inactivated EV71-CA16 Vaccine of Comparative Example 2:

The bivalent inactivated EV71-CA16 vaccine was prepared according to the dilute preparation method of “Comparative example 2” in Table 1:
the monovalent EV71 stock solution (batch number: TZ201702) was accurately pipetted into a 50 mL centrifuge tube according to the formula in “Table 2”, added with an Al(OH)₃ adjuvant according to the amount in “Table 2”, and the resulting mixture was fully and uniformly mixed, and magnetically stirred at room temperature for 15 min to obtain a monovalent EV71 viral antigen-aluminum adsorption product: the monovalent CA16 stock solution (batch number: GX201702) was then accurately pipetted into the above monovalent EV71 viral antigen-aluminum adsorption product, and the resulting mixture was magnetically stirred at room temperature for 15 min: supplemented with 2 mM PBS buffer, further supplemented a glycine mother liquor according to the amount, and the resulting mixture was fully and uniformly mixed: a resulting uniformly mixed sample was sub-packaged to obtain the bivalent inactivated EV71-CA16 vaccine, where the potencies of the EV71 antigen and the CA16 antigen in the bivalent inactivated EV71-CA16 vaccine were both 200 U/dose; and the vaccine was placed in a refrigerator at 2° C. to 8° C. for later use.

### 4) Step of Preparing the Bivalent Inactivated EV71-CA16 Vaccine of Comparative Example 3:

The bivalent inactivated EV71-CA16 vaccine was prepared according to the dilute preparation method of “Comparative example 3” in Table 1:
the monovalent CA16 stock solution (batch number: GX201702) was accurately pipetted into a 50 mL centrifuge tube according to the formula in “Table 2”, added with an Al(OH)₃ adjuvant according to the amount in “Table 2”, and the resulting mixture was fully and uniformly mixed, and magnetically stirred at room temperature for 15 min to obtain a monovalent CA16 viral antigen-aluminum adsorption product: the monovalent EV71 stock solution (batch number: TZ201702) was then accurately pipetted into the above monovalent CA16 viral antigen-aluminum adsorption product, and magnetically stirred at room temperature for 15 min; supplemented with 2 mM PBS buffer, further supplemented with a glycine mother liquor according to the amount, and the resulting mixture was fully and uniformly mixed: a resulting uniformly mixed sample was sub-packaged to obtain the bivalent inactivated EV71-CA16 vaccine, where the potencies of the EV71 antigen and the CA16 antigen in the bivalent inactivated EV71-CA16 vaccine were both 200 U/dose; and the vaccine was placed in a refrigerator at 2° C. to 8° C. for later use.

### Comparative Example 4

A method for preparing a monovalent inactivated EV71 vaccine, involving the steps of:
1. The formula of the adjuvant of the monovalent inactivated EV71 vaccine was as shown in Table 3:

TABLE 3
Formula of monovalent inactivated EV71
vaccine (0.5 mg/mL Al(OH)₃ adjuvant)

| | Initial | | | Final | |
|---|---|---|---|---|---|
| | concentration | | Amount | concentration | |
| Component | (mL⁻¹) | | (mL) | (mL⁻¹) | |
| Monovalent EV71 | 5928 | U | 3.37 | 1000 | U |
| stock solution | | | | | |
| Al(OH)₃ | 13.5 | mg | 0.74 | 0.5 | mg |
| PBS buffer | 2 | mM | 15.39 | / | |
| Glycine | 120 | mg | 0.5 | 3 | mg |

| | | |
|---|---|---|
| Total volume | 20 | / |

2. Step of preparing the monovalent inactivated EV71 vaccine:
   the monovalent EV71 stock solution (batch number: TZ201702) was accurately pipetted into a 50 mL centrifuge tube according to the formula in “Table 3”, added with an Al(OH)₃ adjuvant according to the amount, and the resulting mixture was magnetically stirred at room temperature for 30 min; supplemented with 2 mM PBS buffer, further supplemented with a glycine mother liquor according to the amount, and the resulting mixture was fully and uniformly mixed: a resulting uniformly mixed sample was sub-packaged to obtain the monovalent inactivated EV71 vaccine, where the potency of the EV71 antigen in the monovalent inactivated EV71-CA16 vaccine was 200 U/dose; and the vaccine was placed in a refrigerator at 2° C. to 8° C. for later use.

### Comparative Example 5

A method for preparing a monovalent inactivated CA16 vaccine, involving the steps of:
1. The formula of the adjuvant of the monovalent inactivated CA16 vaccine was as shown in Table 4:

TABLE 4
Formula of monovalent inactivated CA16
vaccine (0.5 mg/mL Al(OH)₃ adjuvant)

| | Initial | | | Final | |
|---|---|---|---|---|---|
| | concentration | | Amount | concentration | |
| Component | (mL⁻¹) | | (mL) | (mL⁻¹) | |
| Monovalent CA16 | 4658 | U | 4.29 | 1000 | U |
| stock solution | | | | | |
| Al(OH)₃ | 13.5 | mg | 0.74 | 0.5 | mg |
| PBS buffer | 2 | mM | 14.47 | / | |
| Glycine | 120 | mg | 0.5 | 3 | mg |

| | | |
|---|---|---|
| Total volume | 20 | / |

2. Step of preparing the monovalent inactivated CA16 vaccine:
   the monovalent CA16 stock solution (batch number: GX201702) was accurately pipetted into a 50 mL centrifuge tube according to the formula in “Table 3”, added with an Al(OH)₃ adjuvant according to the amount, and the resulting mixture was magnetically stirred at room temperature for 30 min: supplemented with 2 mM PBS buffer, further supplemented with a glycine mother liquor according to the amount, and the resulting mixture was fully and uniformly mixed; a resulting uniformly mixed sample was sub-packaged to obtain the monovalent inactivated CA16 vaccine, where the potency of the CA16 antigen in the monovalent inactivated CA16 vaccine was 200 U/dose; and the vaccine was placed in a refrigerator at 2° C. to 8° C. for later use.

### Comparative Example 6

A method for preparing an adjuvant control sample, involving the steps of:
1. The formula of the adjuvant control sample was as shown in Table 5:

TABLE 5
Formula of adjuvant control (0.5 mg/mL Al(OH)₃ adjuvant)

| | Initial concentration | | Amount | Final concentration | |
|---|---|---|---|---|---|
| Component | (mL⁻¹) | | (mL) | (mL⁻¹) | |
| Al(OH)₃ | 13.5 | mg | 0.74 | 0.5 | mg |
| PBS buffer | 2 | mM | 18.76 | / | |
| Glycine | 120 | mg | 0.5 | 3 | mg |

| | | |
|---|---|---|
| Total volume | 20 | / |

2. Step of preparing the adjuvant control sample:
   an Al(OH)₃ adjuvant, 2 mM PBS buffer and a glycine mother liquor were added according to the formula in “Table 5”, and fully and uniformly mixed to prepare the adjuvant control sample, and the adjuvant control sample was placed in a refrigerator at 2° C. to 8° C. for later use.

### Application Example 1

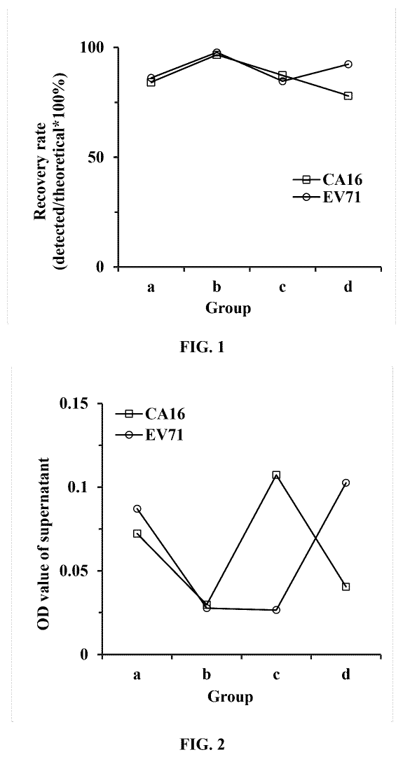

An ELISA antigen content detection kit (EV71 antigen content detection kit (batch number: 20190703, source: Institute of Medical Biology, Chinese Academy of Medical Sciences) and a CA16 antigen content detection kit (batch number: 20191120, source: Aimei Convac BioPharm (Jiangsu) Co., Ltd., Research and Development Department) were used to detect the content of the EV71 and CA16 antigens and the absorbance (OD450 nm) of the free antigens in the experimental samples of Example 1 and Comparative examples 1 to 6. The results are as shown in Tables 6 to 7 and FIGS. 1 to 2, where in FIGS. 1 to 2, a, b, c and d in the abscissa sequentially represent Comparative example 1, Example 1, Comparative example 2 and Comparative example 3.

TABLE 6
Recovery rates of antigens in experimental samples
of Example 1 and Comparative examples 1 to 6

Recovery rate
(detected/theoretical*100%)

| Group | CA16 | EV71 |
|---|---|---|
| Example 1 | 96.67% | 97.76% |
| Comparative example 1 | 84.21% | 86.15% |
| Comparative example 2 | 87.36% | 84.63% |
| Comparative example 3 | 78.05% | 92.34% |
| Comparative example 4 | / | 98.51% |
| Comparative example 5 | 95.09% | / |
| Comparative example 6 | / | / |

Note:
Theoretical value in Table 6 is 1000 U/mL.

TABLE 7
OD values of free antigens in supernatants after centrifugation
of experimental samples of Example 1 and Comparative
examples 1 to 6, as detected by ELISA

OD value of supernatant

| Group | CA16 | EV71 |
|---|---|---|
| Example 1 | 0.0298 | 0.0277 |
| Comparative example 1 | 0.0722 | 0.0871 |
| Comparative example 2 | 0.1073 | 0.0266 |
| Comparative example 3 | 0.0405 | 0.1026 |
| Comparative example 4 | / | 0.0316 |
| Comparative example 5 | 0.0335 | / |
| Comparative example 6 | 0.0213 | 0.0269 |

On the basis of Table 6 and FIG. 1, it can be concluded that the recovery rates of both EV71 and CA16 viral antigens in the experimental sample of Example 1 are both higher than those of Comparative examples 1 to 3, and are both greater than 95%; and except that the recovery rate of the EV71 antigen in Comparative example 3 is 92.34%, the recovery rates of both EV71 and CA16 viral antigens in Comparative examples 1 to 3 are both less than 90%, indicating that using the preparation method of the present disclosure, the recovery rates of the EV71 and CA16 antigens in the bivalent inactivated EV71-CA16 vaccine can reach a high level.

On the basis of Table 7 and FIG. 2, it can be concluded that the OD values of the EV71 and CA16 antigens in the experimental sample of Example 1 are both lower than those in Comparative examples 1 to 3, indicating the best effect of adsorbing the antigens in Example 1.

### Application Example 2

The immunogenicity of each of the experimental samples of Example 1 and Comparative examples 1 to 6 was detected

BALB/c mice (the mice were randomly assigned into a total of 7 groups according to sex and body weight (18 to 22 g), with 10 mice per group including 5 female mice and 5 male mice) were intradermally immunized, in which the 7 groups of mice sequentially corresponded to the bivalent inactivated EV71-CA16 vaccines of Example 1 and Comparative examples 1 to 3, the monovalent inactivated EV71 vaccine of Comparative example 4, the monovalent inactivated CA16 vaccine of Comparative example 5 and the adjuvant control sample of Comparative example 6, respectively.

The experimental samples of Example 1 and Comparative examples 1 to 6 were all administered via intradermal injection at an administration dose of 0.2 mL/animal (intradermal administration at multiple points in the medial side of forelimb and hindlimb, 50 μL/point), with the administration procedure being performed on day 0 and day 28. The animals were observed once a day before administration and within one week after administration to observe the number of survivors, and the diet and activity status of the animals. The body weights were determined once a week before and after administration; and venous blood was collected from all animals on day 28 and day 56 day after the first administration, respectively, the serum was isolated, and the potency of the neutralizing antibody was detected using microneutralization assay.

The results imply that:

1. Observation on the survival and survival status of experimental animals: during the experiment, the mice in 7 groups have normal mobility, normal appearance of fur, and no death.

2. Change in the body weight of experimental animals: during the experiment, the mice in 7 groups all have normal body weight.

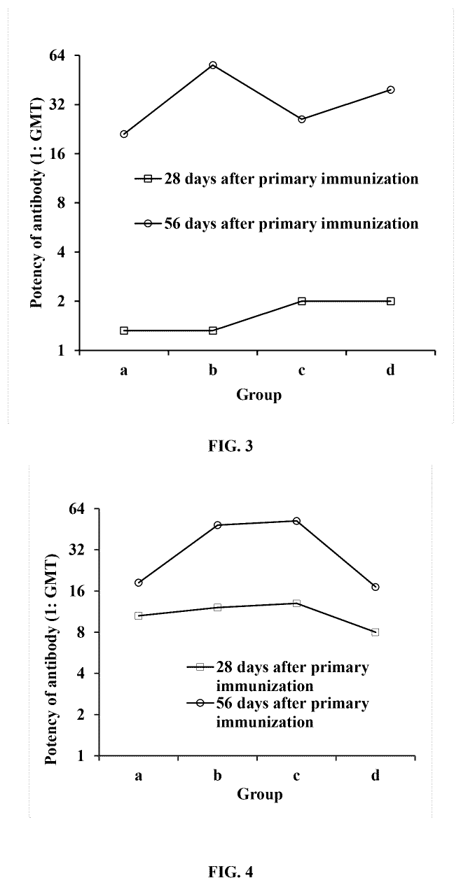

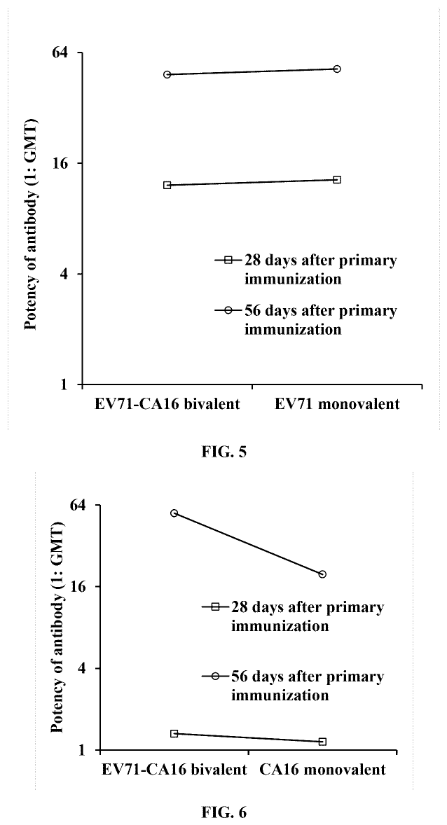

3. The detection results of the potency of the neutralizing antibodies in sera are as shown in Tables 8 to 10 and FIGS. 3 to 6, where in FIGS. 3 to 4, a, b, c and d in the abscissa sequentially represent the bivalent inactivated EV71-CA16 vaccines of Comparative example 1, Example 1, Comparative example 2 and Comparative example 3; and in FIGS. 5 to 6, the EV71-CA16 bivalent, EV71 monovalent and CA16 monovalent in the abscissa sequentially represent the bivalent inactivated EV71-CA16 vaccine of Example 1, the monovalent inactivated EV71 vaccine of Comparative example 4 and the monovalent inactivated CA16 vaccine of Comparative example 5.

TABLE 8
Statistics on the potencies of neutralizing antibodies (GMT
values) in mice immunized with bivalent inactivated EV71-
CA16 vaccines of Example 1 and Comparative examples 1 to 3

| | Blood collection time (after primary immunization) | | | |
|---|---|---|---|---|
| | Day 28 | | Day 56 | |
| Group | Anti-EV71 | Anti-CA16 | Anti-EV71 | Anti-CA16 |
| Example 1 | 12.12573253 | 1.319507911 | 48.50293013 | 55.71523605 |
| Comparative | 10.55606329 | 1.319507911 | 18.37917368 | 21.11212657 |
| example 1 | | | | |
| Comparative | 12.99603834 | 2 | 51.98415337 | 25.99207668 |
| example 2 | | | | |
| Comparative | 8 | 2 | 17.1483754 | 39.39662123 |
| example 3 | | | | |

On the basis of Table 8 and FIGS. 3 to 4, it can be concluded that the high-potency immune serum against EV71 and the high-potency immune serum against CA16 both can be induced in the mice immunized with the bivalent inactivated EV71-CA16 vaccine of Example 1, the antibody against EV71 and the antibody against CA16 which are induced by the bivalent inactivated EV71-CA16 vaccine of Comparative example 1 both show a lower potency, while with regard to the bivalent inactivated EV71-CA16 vaccines of Comparative example 2 and Comparative example 3, the potency of the antibody against later added stock solution is lower than that of the antibody against former added stock solution.

TABLE 9
Statistics on the potencies of neutralizing antibodies
(GMT values) against EV71 after immunization with
vaccines of Example 1 and Comparative example 4

Blood collection time (after primary
immunization)

| | 28 days after | 56 days after |
|---|---|---|
| | primary | primary |
| Group | immunization | immunization |
| Example 1 | 12.12573253 | 48.50293013 |
| Comparative | 12.99603834 | 51.98415337 |
| example 4 | | |

On the basis of Table 9 and FIG. 5, it can be concluded that compared single-dose immunization and two-dose immunization with the bivalent inactivated EV71-CA16 vaccine of Example 1 with single-dose immunization and two-dose immunization with the monovalent inactivated EV71 vaccine of Comparative example 4, the geometric means of the neutralizing antibodies against EV71 are similar, indicating that the level of the neutralizing antibody against EV71 produced by immunization with the bivalent inactivated EV71-CA16 vaccine is consistent with that of the neutralizing antibody against EV71 produced by immunization with the monovalent inactivated EV71 vaccine.

TABLE 10
Statistics on the potencies of neutralizing antibodies
(GMT values) against CA16 after immunization with
vaccines of Example 1 and Comparative example 5

Blood collection time (after primary
immunization)

| | 28 days after | 56 days after |
|---|---|---|
| | primary | primary |
| Group | immunization | immunization |
| Example 1 | 1.319507911 | 55.71523605 |
| Comparative | 1.148698355 | 19.69831061 |
| example 5 | | |

On the basis of Table 10 and FIG. 6, it can be concluded that both single-dose immunization with the bivalent inactivated EV71-CA16 vaccine of Example 1 and single-dose immunization with the monovalent inactivated CA16 vaccine of Comparative example 5 are negative for neutralizing antibodies against CA16, and the level of the neutralizing antibody against CA16 after two-dose immunization with the bivalent inactivated EV71-CA16 vaccine of Example 1 is significantly higher than that after two-dose immunization with the monovalent inactivated CA16 vaccine of Comparative example 5, indicating that the bivalent inactivated EV71-CA16 vaccine prepared in Example 1 of the present disclosure can significantly increase the level of the neutralizing antibody against CA16 in the sera of animals and improve the immunization effectiveness of the CA16 antigen.

### Example 2

The bivalent inactivated EV71-CA16 vaccine was prepared according to the method in Example 1, except that the concentration of Al(OH)₃ adjuvant was 0.25 mg/mL.

### Example 3

The bivalent inactivated EV71-CA16 vaccine was prepared according to the method in Example 1, except that the concentration of Al(OH)₃ adjuvant was 0.75 mg/mL.

### Example 4

The bivalent inactivated EV71-CA16 vaccine was prepared according to the method in Example 1, except that the concentration of Al(OH)₃ adjuvant was 1 mg/mL.

### Comparative Example 7

The bivalent inactivated EV71-CA16 vaccine was prepared according to the method in Example 1, except that the concentration of Al(OH)₃ adjuvant was 0 mg/mL.

### Application Example 3

With reference to the method in Application example 1, the content of the EV71 and CA16 antigens and the absorbance (OD450 nm) of free antigens in the experimental samples of Examples 2 to 4 and Comparative example 7 were detected. The results are as shown in Tables 11 to 12.

TABLE 11
Recovery rates of antigens in bivalent inactivated EV71-CA16
vaccines of Examples 2 to 4 and Comparative example 7

| | |
|---|---|
| | Recovery rate (detected/ |
| Concentration of | theoretical*100%) |

| No. | adjuvant (mg/mL) | EV71 | CA16 |
|---|---|---|---|
| Comparative | 0 | 98.31% | 83.14% |
| example 7 | | | |
| Example 2 | 0.25 | 97.64% | 95.47% |
| Example 3 | 0.75 | 98.03% | 97.28% |
| Example 4 | 1 | 97.34% | 98.73% |

TABLE 12
OD values of supernatants of bivalent inactivated
EV71-CA16 vaccines of Examples 2 to 4 and Comparative
example 7, as detected by ELISA

| | |
|---|---|
| Concentration of | OD value of supernatant |

| No. | adjuvant (mg/mL) | EV71 | CA16 |
|---|---|---|---|
| Comparative | 0 | 2.9851 | 2.8010 |
| example 7 | | | |
| Example 2 | 0.25 | 0.1843 | 0.1020 |
| Example 3 | 0.75 | 0.0306 | 0.0497 |
| Example 4 | 1 | 0.0299 | 0.0518 |

With reference to the method in Application example 2, mice were immunized with the bivalent inactivated EV71-CA16 vaccines of Examples 2 to 4 and Comparative example 7, and the potencies of neutralizing antibodies (GMT values) in the immunized mice were subjected to determination and statistics. The results are as shown in Table 13.

TABLE 13
Statistical data of the potencies of neutralizing antibodies
in the sera of mice immunized with bivalent inactivated EV71-
CA16 vaccines of Examples 2 to 4 and Comparative example 7

| | Concentration | Blood collection time (after primary immunization) | | | |
|---|---|---|---|---|---|
| | of adjuvant | Day 28 | | Day 56 | |
| No. | (mg/mL) | Anti-EV71 | Anti-CA16 | Anti-EV71 | Anti-CA16 |
| Comparative | 0 | 1.866065983 | 1.515716567 | 18.37917368 | 21.11212657 |
| example 1 | | | | | |
| Example 2 | 0.25 | 5.278031643 | 2 | 36.75834736 | 32 |
| Example 3 | 0.75 | 17.1483754 | 5.278031643 | 45.254834 | 55.71523605 |
| Example 4 | 1 | 14.92852786 | 6.498019171 | 59.71411146 | 51.98415337 |

On the basis of Tables 11 to 13 in combination with the data in Application examples 1 to 2, it can be concluded that: when the concentrations of Al(OH)₃ adjuvant is 0.25 to 1.0 mg/mL, the recovery rates of the antigens in each group all reach 95%, and a significant level of neutralizing antibody can be induced in all mice on day 56 after primary immunization.

### Example 5

The bivalent inactivated EV71-CA16 vaccine was prepared according to the method in Example 1, except that the concentration of glycine was 5 mg/mL.

### Example 6

The bivalent inactivated EV71-CA16 vaccine was prepared according to the method in Example 1, except that the concentration of glycine was 10 mg/mL.

### Comparative Example 8

The bivalent inactivated EV71-CA16 vaccine was prepared according to the method in Example 1, except that the concentration of glycine was 0 mg/mL.

### Comparative Example 9

The bivalent inactivated EV71-CA16 vaccine was prepared according to the method in Example 1, except that the concentration of glycine was 1 mg/mL.

### Comparative Example 10

The bivalent inactivated EV71-CA16 vaccine was prepared according to the method in Example 1, except that the protectant was trehalose and the concentration of the trehalose was 1 mg/mL.

### Comparative Example 11

The bivalent inactivated EV71-CA16 vaccine was prepared according to the method in Comparative example 10, except that the concentration of trehalose was 3 mg/mL.

### Comparative Example 12

The bivalent inactivated EV71-CA16 vaccine was prepared according to the method in Comparative example 10, except that the concentration of trehalose was 5 mg/mL.

### Comparative Example 13

The bivalent inactivated EV71-CA16 vaccine was prepared according to the method in Comparative example 10, except that the concentration of trehalose was 10 mg/mL.

### Comparative Example 14

The bivalent inactivated EV71-CA16 vaccine was prepared according to the method in Example 1, except that the protectant was sucrose and the concentration of the sucrose was 1 mg/mL.

### Comparative Example 15

The bivalent inactivated EV71-CA16 vaccine was prepared according to the method in Comparative example 14, except that the concentration of sucrose was 3 mg/mL.

### Comparative Example 16

The bivalent inactivated EV71-CA16 vaccine was prepared according to the method in Comparative example 14, except that the concentration of sucrose was 5 mg/mL.

### Comparative Example 17

The bivalent inactivated EV71-CA16 vaccine was prepared according to the method in Comparative example 14, except that the concentration of sucrose was 10 mg/mL.

### Application Example 4

With reference to the method in Application example 1, the content of the EV71 and CA16 antigens and the absorbance (OD450 nm) of free antigens in the experimental samples of Examples 1 and 5 to 6 and Comparative examples 8 to 17 were detected. The results are as shown in Tables 14 to 15.

TABLE 14
Recovery rates (%) of EV71 antigen in bivalent
inactivated EV71-CA16 vaccines of Examples 1 and
5 to 6 and Comparative examples 8 to 17 time

| | | Concentration | | | |
|---|---|---|---|---|---|
| | Type of | of protectant | Storage time (37° C.) | | |
| No. | protectant | (mg/mL) | 0 d | 7 d | 14 d |
| Comparative | Glycine | 0 | 96.46 | 89.15 | 64.41 |
| example 8 | | | | | |
| Comparative | | 1 | 98.21 | 91.44 | 78.19 |
| example 9 | | | | | |
| Example 1 | | 3 | 98.09 | 97.29 | 95.16 |
| Example 5 | | 5 | 98.24 | 95.26 | 85.46 |
| Example 6 | | 10 | 98.09 | 94.09 | 85.91 |
| Comparative | | 1 | 98.07 | 85.34 | 71.49 |
| example 10 | | | | | |
| Comparative | Trehalose | 3 | 96.48 | 87.19 | 74.19 |
| example 11 | | | | | |
| Comparative | | 5 | 97.24 | 86.53 | 76.84 |
| example 12 | | | | | |
| Comparative | | 10 | 98.87 | 93.46 | 77.14 |
| example 13 | | | | | |
| Comparative | Sucrose | 1 | 97.54 | 87.49 | 62.84 |
| example 14 | | | | | |
| Comparative | | 3 | 98.06 | 88.14 | 61.43 |
| example 15 | | | | | |
| Comparative | | 5 | 98.87 | 89.71 | 62.47 |
| example 16 | | | | | |
| Comparative | | 10 | 97.06 | 91.73 | 60.47 |
| example 17 | | | | | |

TABLE 15
Recovery rates (%) of CA16 antigen in bivalent
inactivated EV71-CA16 vaccines of Examples
1 and 5 to 6 and Comparative examples 8 to 17

| | | Concentration | | | |
|---|---|---|---|---|---|
| | Type of | of protectant | Storage time (37° C.) | | |
| No. | protectant | (mg/mL) | 0 d | 7 d | 14 d |
| Comparative | Glycine | 0 | 96.46 | 89.15 | 64.41 |
| example 8 | | | | | |
| Comparative | | 1 | 98.21 | 91.44 | 78.19 |
| example 9 | | | | | |
| Example 1 | | 3 | 97.19 | 94.72 | 86.29 |
| Example 5 | | 5 | 98.24 | 95.26 | 85.46 |
| Example 6 | | 10 | 98.09 | 94.09 | 85.91 |
| Comparative | | 1 | 98.07 | 85.34 | 71.49 |
| example 10 | | | | | |
| Comparative | Trehalose | 3 | 96.48 | 87.19 | 74.19 |
| example 11 | | | | | |
| Comparative | | 5 | 97.24 | 86.53 | 76.84 |
| example 12 | | | | | |
| Comparative | | 10 | 98.87 | 93.46 | 77.14 |
| example 13 | | | | | |
| Comparative | | 1 | 97.54 | 87.49 | 62.84 |
| example 14 | | | | | |
| Comparative | Sucrose | 3 | 98.06 | 88.14 | 61.43 |
| example 15 | | | | | |
| Comparative | | 5 | 98.87 | 89.71 | 62.47 |
| example 16 | | | | | |
| Comparative | | 10 | 97.06 | 91.73 | 60.47 |
| example 17 | | | | | |

On the basis of Tables 14 to 15 in combination with the data in Application examples 1 to 2, it can be concluded that: although the recovery rates of the EV71 and CA16 antigens in the samples prepared with different concentrations of protectants all reach 95%, the content of the antigens in the samples in the groups which are stored at 37° C. within 7 d show a trend of slow decrease; and the results of the content of the antigens in the samples stored at 37° C. within 14 d show that the content of the antigens in the sucrose group have the greatest decrease, that the best protective effect on the antigens is provided in the glycine group, with a better protective effect at a concentration of 3 to 10 mg/mL, and that especially when the concentration of glycine reaches 3 mg/mL, the trend of change in the content of antigen is small.

### Example 7

The bivalent inactivated EV71-CA16 vaccine was prepared according to the method in Example 1, except that the bivalent inactivated EV71-CA16 vaccine was prepared according to the formula of the bivalent inactivated EV71-CA16 vaccine in Table 16.

TABLE 16
Bivalent inactivated EV71-CA16 vaccine (100 U/100 U/dose)

| | | | | Final | |
|---|---|---|---|---|---|
| | Initial concentration | | Amount | concentration | |
| Component | (mL⁻¹) | | (mL) | (mL⁻¹) | |
| Monovalent EV71 | 5928 | U | 1.69 | 500 | U |
| stock solution | | | | | |
| Monovalent CA16 | 4658 | U | 2.15 | 500 | U |
| stock solution | | | | | |
| Al(OH)₃ | 13.5 | mg | 0.74 | 0.5 | mg |
| PBS buffer | 2 | mM | 14.92 | / | |
| Glycine | 120 | mg | 0.5 | 3 | mg |

| | | |
|---|---|---|
| Total volume | 20 | / |

### Example 8

The bivalent inactivated EV71-CA16 vaccine was prepared according to the method in Example 1, except that the bivalent inactivated EV71-CA16 vaccine was prepared according to the formula of the bivalent inactivated EV71-CA16 vaccine in Table 17.

TABLE 17
Bivalent inactivated EV71-CA16 vaccine (400 U/400 U/dose)

| | Initial concentration | | Amount | Final concentration | |
|---|---|---|---|---|---|
| Component | (mL⁻¹) | | (mL) | (mL⁻¹) | |
| Monovalent EV71 | 5928 | U | 6.75 | 2000 | U |
| stock solution | | | | | |
| Monovalent CA16 | 4658 | U | 8.59 | 2000 | U |
| stock solution | | | | | |
| Al(OH)₃ | 13.5 | mg | 0.74 | 0.5 | mg |
| PBS buffer | 2 | mM | 3.42 | / | |
| Glycine | 120 | mg | 0.5 | 3 | mg |

| | | |
|---|---|---|
| Total volume | 20 | / |

### Application Example 5

With reference to the method in Application example 1, the content of the EV71 and CA16 antigens and the absorbance (OD450 nm) of free antigens in the experimental samples of Examples 7 to 8 were detected. The results are as shown in Table 18.

TABLE 18
Recovery rates of antigens in bivalent inactivated
EV71-CA16 vaccines of Examples 7 to 8

| | |
|---|---|
| | Recovery rate |
| Dose | (detected/theoretical*100%) |

| | (U/U/dose) | EV71 | CA16 |
|---|---|---|---|
| Example 7 | 100 U/100 U | 99.48 | 98.72 |
| Example 8 | 400 U/400 U | 98.91 | 97.34 |

With reference to the method in Application example 2, mice were immunized with the bivalent inactivated EV71-CA16 vaccines of Examples 7 to 8, and the potencies of neutralizing antibodies (GMT values) in the immunized mice were subjected to determination and statistics. The results are as shown in Table 19.

TABLE 19
Statistical data of the potencies of neutralizing antibodies in the sera of mice
immunized with bivalent inactivated EV71-CA16 vaccines of Examples 7 to 8

| | | Blood collection time (after primary immunization) | | | |
|---|---|---|---|---|---|
| | Dose | Day 28 | | Day 56 | |
| No. | (U/U/dose) | Anti-EV71 | Anti-CA16 | Anti-EV71 | Anti-CA16 |
| Example 7 | 100 U/100 U | 4.59479342 | 6.498019171 | 55.71523605 | 64 |
| Example 8 | 400 U/400 U | 9.18958684 | 8 | 78.79324245 | 90.50966799 |

On the basis of Tables 18 to 19 in combination with the experimental data in Application examples 1 to 2, it can be concluded that: when the content of the EV71 antigen and the content of the CA16 antigen in the bivalent inactivated EV71-CA16 vaccine is 500 to 2000 U/mL, i.e., 100 to 400 U/dose, the recovery rates of the antigens are all ≥95%, and a significant level of antibody can be produced in all mice immunized with the vaccines of various groups.

On the basis of the above examples, it can be concluded that: the bivalent inactivated EV71-CA16 vaccine prepared using the preparation method of the present disclosure can achieve high recovery rates of antigens and high potencies of antibodies in the sera of immunized animals.

Although the examples described above have provided a detailed description of the present disclosure, they are only a part of, rather than all of the examples of the present disclosure. All other examples that can be obtained according to the examples of the present disclosure without involving any inventive effort shall fall within the scope of protection of the present disclosure.

1. A method for preparing a bivalent inactivated EV71-CA16 vaccine, wherein the method comprises the steps of:
separately mixing and adsorbing a monovalent EV71 stock solution and a monovalent CA16 stock solution with an adjuvant to obtain a monovalent EV71-adjuvant adsorption product and a monovalent CA16-adjuvant adsorption product, respectively;
mixing and stirring the monovalent EV71-adjuvant adsorption product and the monovalent CA16-adjuvant adsorption product, and mixing a resulting mixture with a protectant to obtain the bivalent inactivated EV71-CA16 vaccine.

2. The method of claim 1, wherein the adjuvant includes aluminum hydroxide, and a mass concentration of the adjuvant in the bivalent inactivated EV71-CA16 vaccine is 0.25 to 1.0 mg/mL.

3. The method of claim 1, wherein the protectant includes glycine, and a mass concentration of the glycine in the bivalent inactivated EV71-CA16 vaccine is 3 to 10 mg/mL.

4. The method of claim 1, wherein a time of the stirring is 30 min.

5. The method of claim 1, wherein prior to mixing with the protectant, further comprised is: diluting the resulting mixture using a buffer.

6. The method of claim 5, wherein the buffer includes a PBS buffer, and the PBS buffer has a concentration of 2 mM and a pH value of 7.00 to 7.40.

7. The method of claim 1, wherein the monovalent EV71 stock solution and the monovalent CA16 stock solution the is prepared by:
respectively inoculating a virus strain onto monolayer cultured cells for culture to obtain virus solutions, wherein the virus strain includes an Enterovirus type 71 strain or a Coxsackie virus type A16 strain;
performing ultrafiltration, concentration and sterilization on the virus solutions to obtain concentrated virus solutions;
performing purification, inactivation and concentration on the concentrated virus solutions to obtain the monovalent EV71 stock solution and the monovalent CA16 stock solution.

8. The method of claim 7, wherein the inoculation amount of the virus strain is 0.1 to 0.25 MOI; the cultured cells include KMB17 cells.

9. The method of claim 7, wherein the culture is conducted at a temperature of 36.5° C. to 37.5° C. for a time of 36 to 80 h.

10. The method of claim 7, wherein the ultrafiltration is performing ultrafiltration on the virus solution sequentially using a depth filter and a sterilizing filter; the depth filter has a filtration accuracy of 0.45 μm; the sterilizing filter has a filtration accuracy of 0.45 μm.

11. The method of claim 7, wherein the concentration is concentrated for 60 times.

12. The method of claim 1, wherein the method of the inactivation comprises: mixing formaldehyde with the purified virus solution obtained from the purification, and performing a reaction for 12 days.

13. The method of claim 12, wherein the final concentration of the formaldehyde in the purified virus solution is 90 μg/mL.

14. A bivalent inactivated EV71-CA16 vaccine, wherein the bivalent inactivated EV71-CA16 vaccine is prepared using the method of claim 1.

15. The bivalent inactivated EV71-CA16 vaccine of claim 14, wherein the content of the EV71 antigen and the content of the CA16 antigen in the bivalent inactivated EV71-CA16 vaccine are both 500 to 2000 U/mL.

16. The bivalent inactivated EV71-CA16 vaccine of claim 14, wherein the dose of the EV71 antigen and the dose of the CA16 antigen in the bivalent inactivated EV71-CA16 vaccine are both 100 to 400 U/dose.

17. (canceled)

18. The method of claim 9, wherein the inoculation amount of the virus strain is 0.1 to 0.25 MOI; the cultured cells include KMB17 cells.

19. The bivalent inactivated EV71-CA16 vaccine of claim 14, wherein the adjuvant includes aluminum hydroxide, and a mass concentration of the adjuvant in the bivalent inactivated EV71-CA16 vaccine is 0.25 to 1.0 mg/mL.

20. The bivalent inactivated EV71-CA16 vaccine of claim 14, wherein a time of the stirring is 30 min.

21. The bivalent inactivated EV71-CA16 vaccine of claim 14, wherein prior to mixing with the protectant, further comprised is: diluting the resulting mixture using a buffer.
